(19) United States
(12) Patent Application Publication
(10) Pub. No.: US 2024/0091295 A1
(43) Pub. Date: Mar. 21, 2024

(54) PIPER NIGRUM COMPOSITION AND METHOD OF TREATING PAIN

(71) Applicants: Kodimule Shyam PRASAD, Bangalore (IN); Kuluvar Gouthamchandra, Bangalore (IN); Karempudi Venkatakrishna, Bangalore (IN); Kuppam Sundeep, Bangalore (IN); Heggar Sudeep, Bangalore (IN)

(72) Inventors: Kodimule Shyam PRASAD, Bangalore (IN); Kuluvar Gouthamchandra, Bangalore (IN); Karempudi Venkatakrishna, Bangalore (IN); Kuppam Sundeep, Bangalore (IN); Heggar Sudeep, Bangalore (IN)

(21) Appl. No.: 18/226,218

(22) Filed: Jul. 25, 2023

Related U.S. Application Data
(60) Provisional application No. 63/392,268, filed on Jul. 26, 2022.

(51) Int. Cl. A61K 36/67; A61K 31/015; A61P 29/00

(52) U.S. Cl. CPC A61K 36/67; A61K 31/015; A61P 29/00

(57) ABSTRACT

The invention provides a composition comprising an extract of black pepper seeds having β-caryophyllene as a bioactive ingredient. The invention also provides a method of providing pain relief, comprising administering to a subject in need thereof an effective amount of the inventive composition.

### CROSS-REFERENCE TO RELATED APPLICATIONS

This application claims the benefit of provisional application No. 63/392,268 filed Jul. 26, 2022, the entire contents of which are incorporated herein by reference for all purposes.

### FIELD OF INVENTION

The invention is in the field of bioactive botanical preparations. More particularly, the invention is directed to a composition comprising a black pepper seed extract and method of its use in the treatment of pain and providing pain relief.

### BACKGROUND OF THE INVENTION

Pain is a serious health issue with notable socioeconomic concerns affecting work productivity (Cazacu et al. 2015; Fongang et al. 2017). Pain is associated with inflammation which can lead to tissue damage and a loss of functionality (Furtado et al. 2016). Nevertheless, a good number of analgesics such as opioids and non-steroidal anti-inflammatory drugs (NSAIDS) are available in the market, but are associated with undesirable side effects (Tacconelli et al. 2017; Cazacu et al. 2015). This necessitates searching for complementary and alternative medicine which can relieve pain and inflammation, and improve quality of life. In this direction, dietary supplements and herbal extracts have gained significant attention, several of them being used traditionally in ethnomedicine (Reynolds et al. 1995).

Several culinary spices including clove, turmeric, and ginger have been reported to possess analgesic activity (Kamkar Asl et al. 2013; Sahebkar and Henrotin, 2016; Rayati et al. 2017; Tasleem et al. 2014). It is well-known that black pepper (Piper nigrum) contains piperine as a major bioactive constituent that has various pharmacological benefits. The essential oil of Piper nigrum contains terpenes such as β-caryophyllene, limonene, β-pinene and sabinene (Wang et al. 2018).

### BRIEF DESCRIPTION OF DRAWINGS

FIG. 1 shows the GCMS chromatograms of β-caryophyllene in an embodiment of the inventive composition and the reference standard.

FIG. 2 shows the analgesic effect of an embodiment of the inventive composition in acetic acid-induced writhing test in mice.

FIG. 3 shows the effect of pre-treatment with selective antagonists of pain receptors on analgesic activity in comparison to an embodiment of the inventive composition in formalin-induced paw licking model mice.

FIG. 4 shows the effect of pre-treatment with selective antagonists of pain receptors on analgesic activity in comparison to an embodiment of the inventive composition (hot plate test).

FIG. 5 shows the effect of pre-treatment with selective antagonists of pain receptors on analgesic activity in comparison to an embodiment of the inventive composition (tail flick test).

FIG. 6 shows the three-dimensional view of the nociceptive receptors used for the molecular docking studies.

FIG. 7 shows the representative docking pose predictions of β-caryophyllene with the agonist binding sites of nociceptive receptors.

The following detailed description and the accompanying drawings to which it refers are intended to describe some, but not necessarily all, examples and embodiments of the invention. The described embodiments are to be considered in all respects only as illustrative and not restrictive. The contents of this detailed description and the accompanying drawings do not limit the scope of the invention in any way. References to “Viphyllin” in the drawings refer to an embodiment of the inventive composition.

### DETAILED DESCRIPTION

The present invention provides a composition comprising a black pepper seed extract and method of using the composition in the treatment of pain. In some non-limiting embodiments, the extract is obtained from Piper nigrum seeds. The black pepper seed extract can contain terpenes as a bioactive constituent. The black pepper seed extract can contain (3-caryophyllene as a bioactive constituent. The black pepper seed extract can contain between about 10% w/w and about 80% w/w β-caryophyllene. As used herein, the term “about” means the quantity or value that is referenced, or that varies (plus or minus) by up to 2%, 5%, 10%, 15% or 20% of the quantity or value that is referenced. In some embodiments, the black pepper seed extract can consist essentially of β-caryophyllene. Without being limited to any particular theory or mechanism of action, the bioactive constituents of black pepper seed extract impart their therapeutic effect by antagonism of the CB2, TRPV1 and PPARα receptors.

In some embodiments, the composition comprises black pepper seed extract in combination with a carrier or excipient. Suitable carriers and excipients for use with the invention include, but are not necessarily limited to, those disclosed in the following references, the entire contents of which are incorporated herein by reference for all purposes: Remington: The Science and Practice of Pharmacy, 19^{th} Ed (Easton, Pa.: Mack Publishing Company, 1995); Hoover, John E., Remington's Pharmaceutical Sciences, (Easton, Pa.: Mack Publishing Co 1975); Liberman, H. A. and Lachman, L., Eds., Pharmaceutical Dosage Forms (New York, N.Y.: Marcel Decker 1980); and Pharmaceutical Dosage Forms and Drug Delivery Systems, Seventh Ed (Lippincott Williams & Wilkins 1999). In some embodiments, the composition comprises an artificial carrier or excipient. The composition can further comprise fillers, diluents, binders, lubricants, disintegrants, or combinations thereof.

The composition can be formulated as a powder, liquid, pill, tablet, pellet, capsule, thin film, solution, spray, syrup, linctus, lozenge, pastille, chewing gum, paste, vapor, suspension, emulsion, ointment, cream, lotion, liniment, gel, drop, topical patch, buccal patch, bead, gummy, gel, sol, or injection. The composition can be formulated for oral administration. The composition can further comprise vitamins, minerals, amino acids, proteins, carbohydrates, lipids, fatty acids, caffeine, flavorings, sweeteners, preservatives, or combinations thereof. In some embodiments, the composition is combined with an edible substance. The composition can be combined with the edible substance by mixing, formulating, folding, or blending, for example. Edible substances for use with the invention include, but are not necessarily limited to foods, beverages, nutritional supplements, and dietary supplements.

In at least one aspect of the invention, the composition is provided and/or manufactured in bulk. The composition can be provided in bulk for the manufacture of foods, nutritional supplements, nutraceuticals, dietary supplements and/or food supplements for the treatment of pain. Bulk quantities of the composition can be packaged, stored and/or distributed in drums, bags, boxes, containers and the like. Such containers can be configured to prevent or inhibit the oxidation of the bioactive constituents of the composition.

In some embodiments, the invention provides a method of treating pain. As used herein, the phrases “treating pain,” “treatment of pain,” “providing pain relief,” and the like, refer to preventing, arresting, inhibiting the progression of, delaying the onset of, or reducing the sensation of pain in a subject.

The method can be practiced by administering to a mammalian subject in need thereof (e.g. a human) an effective amount of a composition as disclosed herein. The subject can have or be at risk of developing pain associated with the peripheral nociceptive system, the central nociceptive system, or a combination thereof. The subject can have, or be at risk of developing, pain associated with migraines, headaches (including tension-type and cluster headaches), trigeminal neuralgia, herpetic neuralgia, general neuralgias, postherpetic neuralgia, cramping (including menstrual cramping), psychogenic pain, allodynia, carpal tunnel syndrome pain, fibromyalgia pain, arthritis pain (including rheumatoid arthritis and osteoarthritis pain), pain due to sunburn, peripheral neuropathy pain, diabetic neuropathy pain, radicular pain, sciatica, back pain, head and neck pain, hyperalgesia, spontaneous pain, phantom pain, idiopathic pain, pain in the skin and musculoskeletal tissues itching, pain from insect stings and bites, dysesthesia (including thermal and cold dysesthesia), severe or intractable pain, breakthrough pain, persistent pain, tendinitis pain, postsurgical pain, cancer pain, dental pain, muscle aches, pain due to injury including sports and occupational injuries (including burns, abrasions, fractures, sprains, strains, tears, contusions, cuts, and the like).

The composition can be administered systemically and/or locally. The composition can be administered locally, such as the site of pain, for example. Suitable administration routes for the composition include, but are not limited to, auricular, buccal, conjunctival, cutaneous, dental, endocervical, endosinusal, endotracheal, enteral, epidural, extra-amniotic, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal dental, intracoronary, intracorporus cavernosum, intradermal, intradiscal, intraductal, intraduodenal, intradural, intraepidermal, intraesophageal, intragastric, intravaginal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravitreal, laryngeal, nasal, nasogastric, ophthalmic, oral, oropharyngeal, parentera, percutaneous, periarticular, peridural, perineural, periodontal, rectal, inhalation, retrobulbar, soft tissue, subarachnoid, subconjunctival, subcutaneous, sublingual, submucosal, topical, transdermal, transmucosal, transplacental, transtracheal, transtympanic, ureteral, urethral, vaginal, or combinations thereof. The composition can be administered by irrigation, drip, infusion, or topically by a dressing, patch, or bandage that is in contact with the composition. In some preferred embodiments, the composition is administered orally. The composition can be placed on wound dressings, such as bandages, gauze, cotton swabs and balls, wipes, and patches, for example.

Black pepper seed extract for use with the invention can be obtained by extracting one or more bioactive constituents from black pepper seeds using a solvent suitable for extracting the bioactive constituents of the seeds. The black seeds can be Piper nigrum seeds. Suitable, non-limiting solvents for making the black pepper seed extract include, but are not necessarily limited to, alcohol-based solvents aqueous solvents, hydroalcoholic solvents, supercritical fluid solvents, and combinations thereof. Alcohol-based solvents for use with the invention include, but are not necessarily limited to, ethanol, isopropyl alcohol, methanol, and combinations thereof. Black pepper seeds for providing the extract can be fresh, dried, or a combination thereof. The black pepper seeds can be crushed, whole, or powdered.

The present invention is described in detail by the following examples. Example 1 illustrates the quantitative analysis of the inventive composition by gas chromatography-mass spectrometry (GCMS). Example 2 describes the anti-nociceptive effects of the inventive composition and the involvement of pain receptors in its mechanism of action. Example 3 demonstrates the molecular interaction of β-caryophyllene (principal component of the inventive composition) with the agonist-binding sites of the pain receptors.

### Example 1

The characterization of β-caryophyllene in the inventive composition was determined using GC-MS. A capillary gas chromatograph (Shimadzu GC-2030, Japan) equipped with autosampler, and a flame ionization detector (FID) used for the analysis. The separation was achieved using capillary column SH-Rxi-5Sil MS (30 m×0.25 mm×0.25 μm). The injector and detector temperatures were set at 210 and 300° C. respectively. The oven temperature was kept at 285° C. The GC-FID injected volume was 1 μL and the solutions were injected in 1/10 split ratio. The GC oven temperature was programmed with a run time of 12 min was as follows: the column was held initially at 40° C. for 1.0 min, then increased to 285° C. at 20° C./min and held for 6 min. The helium and argon were used as a carrier and collision induced dissociation (CID) gas, respectively. The hydrogen and air flow were set at 50.0 and 300.0 mL/min, respectively. The chromatographic data were recorded and integrated using LabSolutions software.

### Preparation of Standard Solution

Accurately weighed amount of β-caryophyllene standard was taken in 100 mL standard volumetric flask and dissolved in dichloromethane to obtain a concentration of 250 parts per million (ppm). The standard solution was filtered through 0.2μ nylon syringe filter and injected the solution in the column.

### Preparation of Sample Solution

The inventive composition was prepared in dichloromethane to achieve the final concentration of 250 ppm; filtered the sample solution through 0.2μ nylon syringe filter and injected the solution.

### Results

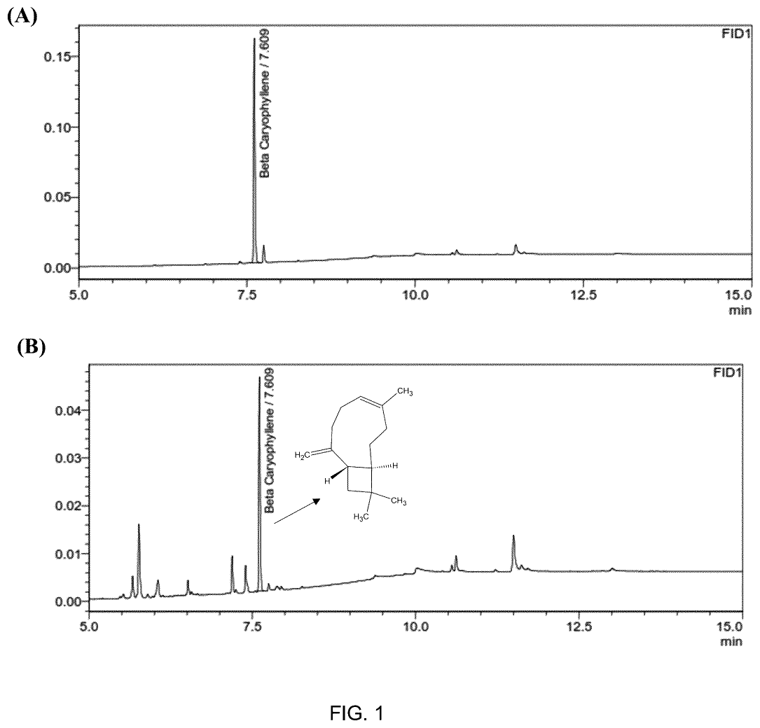

The optimisation of chromatographic condition was achieved by comparing the chromatogram obtained from blank, reference standard, and the inventive composition. The retention time of β-caryophyllene reference standard was found to be 7.609 min. The presence of β-caryophyllene in the inventive composition was confirmed with the matching retention time of 7.609 min without any interferences (FIG. 1).

### Example 2

The herbal composition of the present invention was evaluated for analgesic activity and the involvement of potential nociceptive receptors in its mechanism of action was demonstrated using different experimental pain models.

### Animals

Male Balb/c mice procured from the authorized animal supplier Biogen Laboratory Animal Facility, Bangalore, India were used for the animal experiments. The animals were housed in the animal facility of R&D Center, Vidya Herbs Pvt Ltd under controlled environmental conditions (temperature: 23±2° C., relative humidity: 35-50%, 12 h light/dark cycle). The animals were given commercial pellet diet and water ad libitum. All the animals were allowed to acclimatize to the environmental conditions before the start of experiments. The experimental protocols were approved by the Institutional Animal Ethics Committee (IAEC) of Vidya Herbs Pvt Ltd., Bangalore, India (VHPL/PCL/IAEC/01/2021).

### Acetic Acid-Induced Writhing Test

Twenty-four male mice were randomized to four groups (n=6 in each group): Control group were given normal saline solution while positive control group received reference drug Diclofenac sodium (30 mg/kg). Two test doses of the inventive composition (25 and 50 mg/kg body weight) were used in the test. All the treatments were intraperitoneally injected. 30 min after the respective treatments, the animals were given 10 mL/kg body weight of 1% acetic acid intraperitoneally. The number of writhes were recorded after 10 min of acetic acid administration.

### Formalin-Induced Paw Licking Test

Formalin-induced paw licking test was performed on 36 male mice divided into six groups (6/group). Control group received vehicle while another group received the effective dose of the inventive composition (50 mg/kg body weight). Other groups were intraperitoneally injected with different antagonists viz., capsazepine (0.1 mg/kg body weight), GW6471 (1 mg/kg), AM630 (5 mg/kg), and SR141716A (0.1 mg/kg). 30 min after the antagonist administration, the mice were given the inventive composition 50 mg/kg body weight i.p. Formalin test (Janssen et al. 1963) was conducted thirty minutes after the vehicle/extract treatment. Briefly, the animals were given 20 IA intraplantar injection of a 2.5% formalin solution. The injected paw-licking time (in seconds) was recorded during early (0-5 min) and late phase (25-30 min) after formalin injection.

### Hot Plate Test

Mice were divided into different groups with six animals in each group. Control group was injected with vehicle and the treatment group received the inventive composition (50 mg/kg body weight). The other groups were injected with the respective doses of antagonists as mentioned in previous experimental section. The mice were given the inventive composition 50 mg/kg body weight i.p. 30 min after the antagonist administration. Thirty minutes later, the animals were placed on a thermostatic hot plate with a temperature maintained at 55±2° C. The latency time i.e., the time taken by the mice for initial jump was recorded. To prevent tissue damage, a cutoff time of 60 s was used (Jaffal and Abbas, 2018).

### Tail Flick Test

The animals were randomized into different groups as detailed above. Mice were injected with the vehicle/the inventive composition (50 mg/kg body weight) 30 min after i.p. administration of different antagonists as described previously. After 30 min of extract treatment, tail flick (immersion) test was performed. In brief, tail of each animal (5 cm) was immersed in a water bath maintained at 55±2° C. The reflex time of animal to withdraw the tail was recorded using a stopwatch. The cut off time was kept as 15 s to avoid tissue injury.

### Statistical Analysis

All the experimental data were analyzed by one way ANOVA followed by Tukey's multiple comparison test using GraphPad Prism 9.0 (GraphPad Software, Inc.). The data were presented as mean±SEM. p<0.05 were considered statistically significant.

### Results

### Effect of the Inventive Composition on Acetic Acid-Induced Writhing Response in Mice

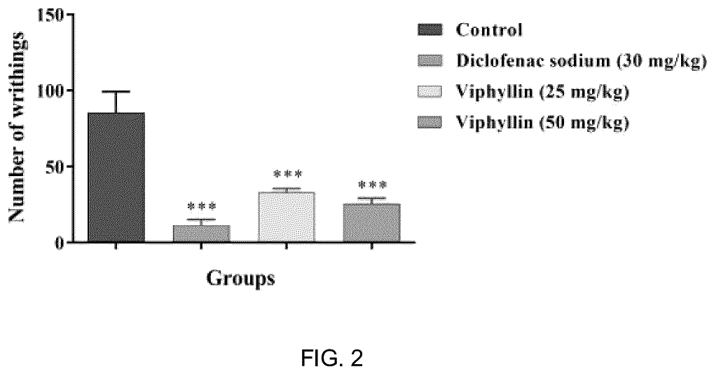

In this study, the anti-nociceptive effect of the inventive composition was initially screened using acetic acid-induced writhing test in mice. FIG. 2 shows the number of writhings recorded in different treatment groups. the inventive composition at the doses of 25 and 50 mg/kg body weight showed significant analgesic activity in dose-dependent fashion (p<0.001). the inventive composition showed 61.12% and 69.97% inhibition in number of writhes at 25 and 50 mg/kg, as compared to control mice. The reference standard diclofenac sodium exhibited the writhing inhibition of 86.55% with respect to control group. Based on these data, we have used 50 mg/kg of the inventive composition for subsequent experiments.

### Involvement of Nociceptive Receptors in the Inventive Composition-Mediated Analgesic Activity

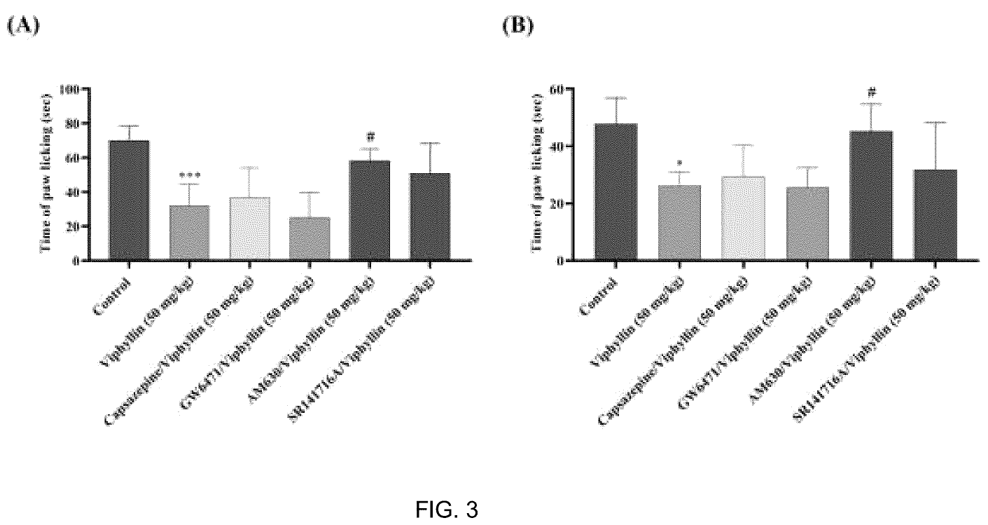

The involvement of different pain receptors in the analgesic action of the inventive composition was investigated. In the early (p<0.001) and late phases (p<0.05) of formalin test, the extract at 50 mg/kg body weight significantly reduced the paw licking time compared to the vehicle treated group. The reduction in the paw licking time of the inventive composition-treated mice was reversed to a significant extent (p<0.05) by the selective CB2 receptor antagonist (AM630) in both early and late phases of formalin test (FIG. 3), as compared to the inventive composition-treated mice. However, there was no significant change in the effect of the inventive composition observed following treatments with antagonists of TRPV1 (capsazepine), PPARα (GW6471) and CB1 (SR141716A).

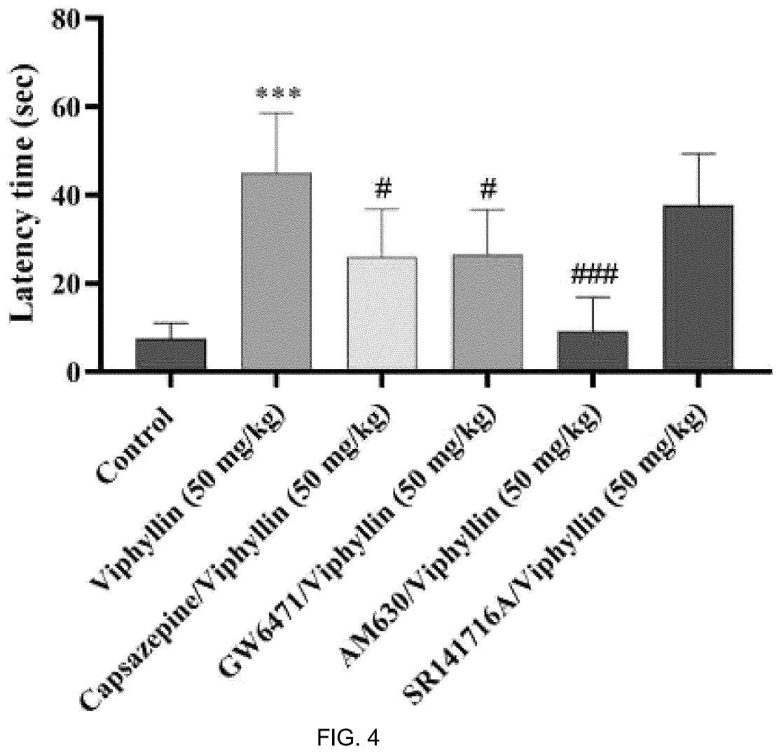

In the hot plate test, the inventive composition treatment markedly enhanced the latency time of mice compared to the vehicle control group (FIG. 4). The extract-mediated analgesic action was significantly reduced using selective TRPV1, PPARα (p<0.05) and CB2 (p<0.001) receptor antagonists. CB1 antagonist did not have any significant influence on the effect of the inventive composition.

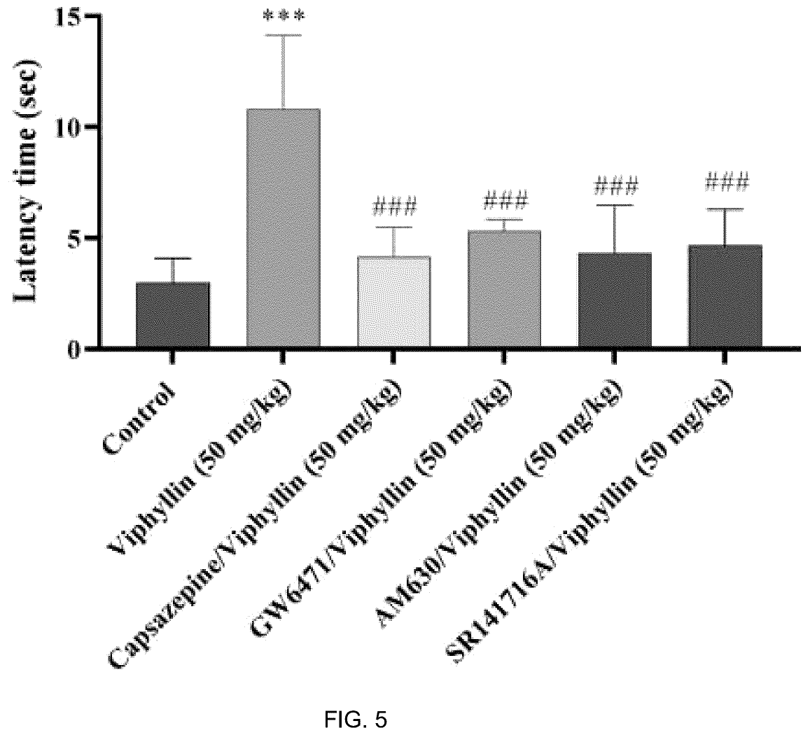

In tail flick test, the inventive composition-treated group demonstrated significantly higher latency time compared to control group (p<0.001). Interestingly, the action of the inventive composition was reversed by antagonists of PPARα, TRPV1, CB2 and CB1 receptors (p<0.001) (FIG. 5).

Collectively, the experimental data obtained using different pain models in mice strongly suggest that the inventive composition has profound analgesic activity, with the involvement of CB2, TRPV1 and PPARα receptors.

### Example 3

Molecular docking study was performed to predict the binding mode of (3-caryophyllene with the agonist-binding sites of the pain receptors: CB′, CB2, PPARα and TRPV1 receptors.

### Protein Structures

The crystal structures of agonist-bound CB-1 (PDB ID: 5XR8), CB-2 (PDB ID: 6PT0), PPARα (PDB ID: 6KXY) receptors and TRPV1 ion channel (PDB ID: 3J5R) were retrieved from PDB database (http://www.rcsb.org/). Chain A of CB1 and PPARα receptors, chain R of CB2 receptor, and chain B of TRPV1 were used for macromolecule preparation. The coordinates of PDB structures were prepared for molecular docking by removing the water ions and ligands using Python molecule viewer.

### Ligand Preparation

The 3D structures of β-caryophyllene were obtained from Pubchem (https://pubchem.ncbi.nlm.nih.gov). The drug-like properties of the molecules were determined using SWISSADME prediction (http://www.swissadme.ch/). The OpenBabel tool was used to minimise energy of natural compounds and 3D coordinates were prepared (http://www.cheminfo.org/).

### Molecular Docking

The crucial amino acid residues actively involved in the agonist binding of the nociceptive receptors were retrieved from the literature (Hua et al. 2017; Gertsch et al. 2008; Yang and Zheng, 2017; Yoshida et al. 2020). AutoDock tool was utilized to generate grids, calculate dock score, and evaluate the conformers of β-caryophyllene interacting in the binding sites of receptors. The grid map parameters were generated with AutoGrid (Table 1). As per genetic algorithm, all the torsions could rotate during docking. The Lamarckian genetic algorithm and the pseudo-Solis and Wets methods were applied for minimization, using default parameters (Rodriguez and Infante, 2011).

TABLE 1
Grid point parameters used for molecular docking of β-caryophyllene
with nociceptive receptor-agonist binding sites.

| | Grid centre coordinates | | | Grid size | | |
|---|---|---|---|---|---|---|
| Receptor | X | y | z | x | y | z |
| CB1 | −43.288 | −157.799 | 307.888 | 40 | 40 | 40 |
| CB2 | 97.135 | 109.16 | 120.83 | 60 | 60 | 60 |
| PPARα | 15.295 | −12.269 | −32.623 | 46 | 40 | 54 |
| TRPV1 | 24.113 | −7.236 | −13.071 | 50 | 50 | 50 |

### Results

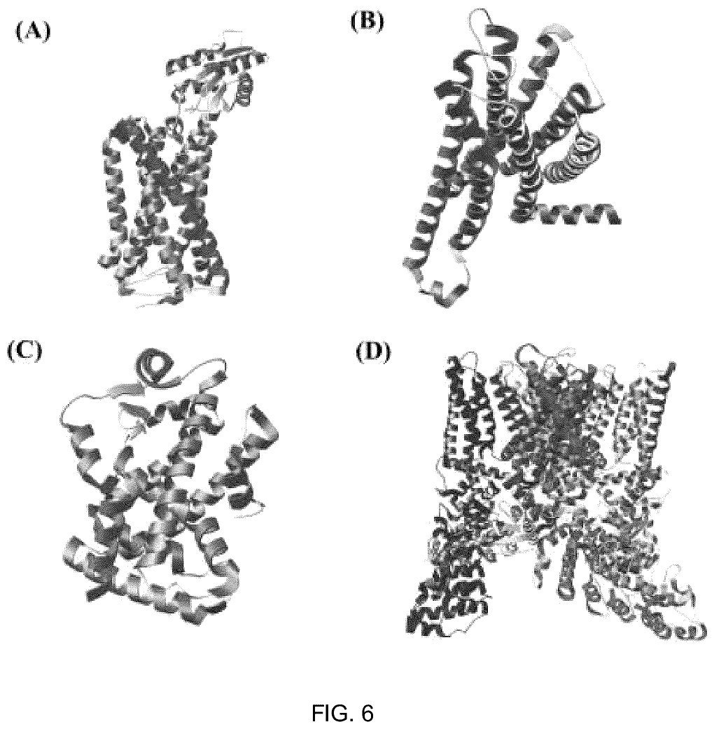

Chain A of CB1 and PPARα receptors, chain R of CB2 receptor, and chain B of TRPV1 were used for docking studies (FIG. 6). Table 2 shows the docking scores of β-caryophyllene bound in the agonist-binding sites of the pain receptors.

TABLE 2
Docking score of interaction of β-caryophyllene with agonist binding sites of different pain receptors

| | Binding | | Inhibitory | | |
|---|---|---|---|---|---|
| | energy | Ligand | constant | | |
| Receptor | kJ/mol | efficiency | (μM) | Vdw_hb_desolv_energy | Interactions |
| Cannabinoid | −7.83 | −0.52 | 1.81 | −7.84 | Ile186, Ile110, Trp194, |
| receptor 2 (CB2) | | | | | Phe281, Val113, Phe183, |
| | | | | | Thr114 |
| Cannabinoid | −5.16 | −0.34 | 165.22 | −5.16 | Phe191, Gly194, Gly 195, |
| receptor 1 (CB1) | | | | | Ala198, Ile245, Val249 |
| PPAR-α | −6.66 | −0.44 | 13.05 | −6.67 | Ile241, Leu247, Glu251, |
| | | | | | Leu254, Arg271, Ile272, |
| | | | | | Cys275, Val332, Ala333 |
| TRPV1 | −7.58 | −0.51 | 2.8 | −7.57 | Phe488, Arg491, Gly492, |
| | | | | | Glu513, Phe516, Tyr554 |

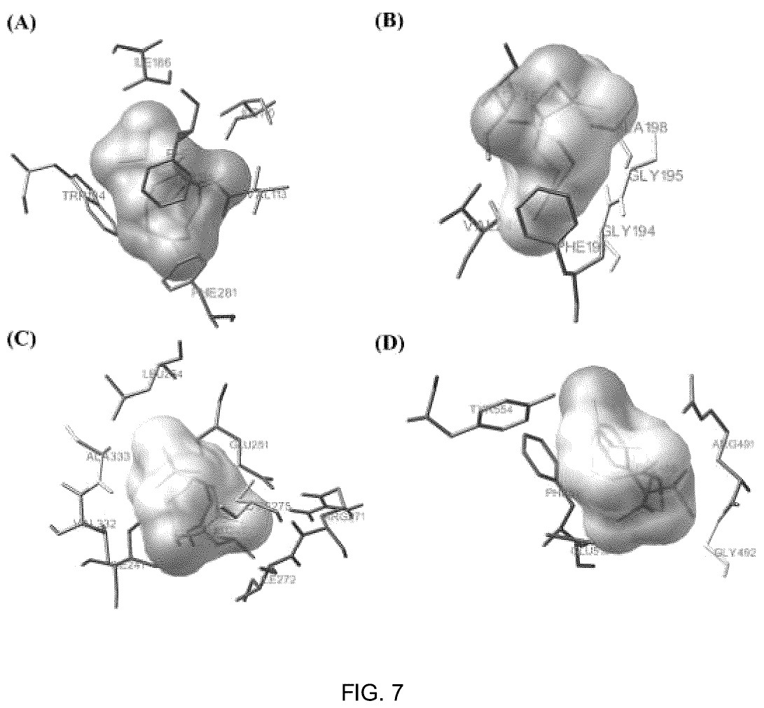

β-caryophyllene showed an appreciable interaction pattern in the orthosteric pocket of CB2 with a binding energy of −7.83 kJ/mol and Ki of 1.81 μM. Due to the structural constraints from the nine-membered ring, β-caryophyllene demonstrated only four geometries during docking. The molecule closely interacted with hydrophobic residues Trp194, and Phe183 and Ile186 of CB2 binding site (FIG. 7A). These residues have been reported to be crucial to function in ligand binding (Xing et al. 2020; Feng et al. 2014). In addition, (3-caryophyllene had profound interactions with Ile110, Thr114, Val113 and Phe281. (3-caryophyllene had a moderate binding with CB1 receptor binding site. The best docking pose showed a binding energy of −5.16 kJ/mol (Ki=165.22 μM). β-caryophyllene did not interact with any of the key residues of CB1 (FIG. 7B). The best scoring pose of β-caryophyllene with PPAR-α putative binding site (Ki=13.05 μM) showed key interactions including Ile241, Ile272, and Ala333, and the lowest binding energy of −6.66 kJ/mol. β-caryophyllene showed several molecular interactions with the amino acid residues of the binding pocket (FIG. 7C). The docking results of β-caryophyllene against TRPV1 ion channel showed the lowest binding energy value of −7.58 kJ/mol (Ki=2.8 μM). β-caryophyllene engaged in interaction with Phe488, Arg491, Gly492, Glu513, Phe516, and Tyr554 residues in the capsaicin-binding pocket of TRPV1 (FIG. 7D).

### REFERENCES

1. Cazacu I, Mogosan C, Loghin F. Safety issues of current analgesics: an update. Clujul Med 2015; 88(2):128-136.
2. Fongang A L M, Laure Nguemfo E, Djouatsa Nangue Y, Bogning Zangueu C, Fouokeng Y, Azebaze A G B, Jose Llorent-Martinez E, Cordova M L F, Bertrand Dongmo A, Vierling W. Antinociceptive and anti-inflammatory effects of the methanolic stem bark extract of Antrocaryon klaineanum Pierre (Anacardiaceae) in mice and rat. J Ethnopharmacol 2017; 203: 11-19.
3. Furtado A A, Torres-Rego M, Lima M C, Bitencourt M A, Estrela A B, Souzada Silva N, da Silva Siqueira E M, Tomaz J C, Lopes N P, Silva-Junior A A, Zucolotto S M, Fernandes-Pedrosa M F. Aqueous extract from Ipomoeaasarifolia (Convolvulaceae) leaves and its phenolic compounds have anti-inflammatory activity in murine models of edema, peritonitis and air-pouch inflammation. J Ethnopharmacol 2016; 192: 225-235.
4. Tacconelli S, Bruno A, Grande R, Ballerini P, Patrignani P. Nonsteroidalanti-inflammatory drugs and cardiovascular safety—translating pharmacologicaldata into clinical readouts. Expert opinion on drug safety 2017; 16: 791-807.
5. Reynolds J F, Noakes T D, Schwellnus M P, Windt A, Bowerbank P. Non-steroidal anti-inflammatory drugs fail to enhance healing of acute hamstring injuries treated with physiotherapy. S Afr Med J. 1995; 85(6):517-22.
6. Kamkar Asl M, Nazariborun A, Hosseini M. Analgesic effect of the aqueous and ethanolic extracts of clove. Avicenna J Phytomed. 2013; 3(2): 186-192.
7. Sahebkar A, Henrotin Y. Analgesic efficacy and safety of curcuminoids in clinical practice: A systematic review and meta-analysis of randomized controlled trials, Pain Medicine 2016; 17(6): 1192-1202, https://doi.org/10.1093/pm/pnv024
8. Rayati F, Hajmanouchehri F, Najafi E. Comparison of anti-inflammatory and analgesic effects of Ginger powder and Ibuprofen in postsurgical pain model: A randomized, double-blind, case-control clinical trial. Dent Res J (Isfahan). 2017; 14(1):1-7. doi:10.4103/1735-3327.201135
9. Tasleem F, Azhar I, Ali S N, Perveen S, Mahmood Z A. Analgesic and anti-inflammatory activities of Piper nigrum L. Asian Pac J Trop Med. 2014; 7S1:S461-8. doi: 10.1016/S1995-7645(14)60275-3.
10. Wang M, Chittiboyina A, Parcher J, Ali Z, Ford P. Zhao J, Avula B, Wang Y, and Khan I. Piper nigrum oil—Determination of selected terpenes for quality evaluation. Planta Med 2018; 17: 84
11. Klauke A L, Racz I, Pradier B, Markert A, Zimmer A M, Gertsch J, Zimmer A. The cannabinoid CB2 receptor-selective phytocannabinoid beta-caryophyllene exerts analgesic effects in mouse models of inflammatory and neuropathic pain. Eur Neuropsychopharmacol 2014; 24(4): 608-620.
12. Jaffal S M, Abbas M A. Antinociceptive action of Achillea biebersteinii methanolic flower extract is mediated by interaction with cholinergic receptor in mouse pain models. Inflammopharmacol 2018; 27(5):961-968
13. Janssen P A. The inhibitory effect of fentanyl and other morphine-like analgesics on the warm water induced tactile withdrawal reflex in rat. Arzneimittelforschung 1963; 13:502-507
14. Hua T, Vemuri K, Nikas S P, Laprairie R B, Wu Y, Qu L, Pu M, Korde A, Jiang S, Ho J R, Han G W, Ding K. Li X, Liu H, Hanson M A, Zhao S, Bohn L M, Makriyannis A, Stevens R C, Liu Z J. Crystal structures of agonist-bound human cannabinoid receptor CB1. Nature. 2017; 547(7664): 468-471. doi: 10.1038/nature23272.
15. Gertsch J, Leonti M, Raduner S, et al. Beta-caryophyllene is a dietary cannabinoid. Proc Natl Acad Sci USA. 2008; 105(26):9099-104. doi: 10.1073/pnas.0803601105.
16. Yang F, Zheng J. Understand spiciness: mechanism of TRPV1 channel activation by capsaicin. Protein Cell. 2017; 8(3): 169-177. doi:10.1007/s13238-016-0353-7
17. Yoshida T, Oki H, Doi M, Fukuda S, Yuzuriha T, Tabata R, Ishimoto K, Kawahara K, Ohkubo T, Miyachi H, Doi T, Tachibana K. Structural Basis for PPARα Activation by 1H-pyrazolo-[3,4-b]pyridine Derivatives. Sci Rep. 2020 May 6; 10(1):7623. doi: 10.1038/s41598-020-64527-x. PMID: 32376995.
18. Rodriguez A, Infante D. Characterization in silico of flavonoids biosynthesis in Theobroma cacao L. Net Biol 2011; 1: 34-45.
19. Xing C, Zhuang Y, Xu T H, et al. Cryo-EM Structure of the Human Cannabinoid Receptor CB2-Gi Signaling Complex. Cell. 2020; 20; 180(4): 645-654.e13. doi: 10.1016/j.cell.2020.01.007.
20. Feng Z, Alqarni M H, Yang P, et al. Modeling, molecular dynamics simulation, and mutation validation for structure of cannabinoid receptor 2 based on known crystal structures of GPCRs. J Chem Inf Model. 2014; 54: 2483-2499.

1. A composition for pain relief, comprising between about 10% w/w and about 80% w/w a β-caryophyllene.

2. The composition of claim 1, wherein said composition comprises Piper nigrum seed extract.

3. The composition of claim 1, wherein said composition comprises Piper nigrum seed extract having between about 10% w/w and about 80% w/w a β-caryophyllene.

4. The composition of claim 1, wherein said composition comprises an artificial carrier or excipient.

5. The composition of claim 1, wherein said composition is in form selected from a powder, liquid, pill, tablet, pellet, capsule, thin film, solution, spray, syrup, linctus, lozenge, pastille, chewing gum, paste, vapor, suspension, emulsion, ointment, cream, lotion, liniment, gel, drop, topical patch, buccal patch, bead, gummy, gel, sol and injection.

6. A method of providing pain relief, comprising administering to a subject in need thereof an effective amount of a composition comprising between about 10% w/w and about 80% w/w a β-caryophyllene.

7. The method of claim 6, wherein said composition comprises Piper nigrum seed extract.

8. The method of claim 6, wherein said composition comprises Piper nigrum seed extract having between about 10% w/w and about 80% w/w a β-caryophyllene.

9. The method of claim 6, wherein said composition comprises an artificial carrier or excipient.

10. The method of claim 6, wherein said composition is in form selected from a powder, liquid, pill, tablet, pellet, capsule, thin film, solution, spray, syrup, linctus, lozenge, pastille, chewing gum, paste, vapor, suspension, emulsion, ointment, cream, lotion, liniment, gel, drop, topical patch, buccal patch, bead, gummy, gel, sol and injection.

11. The method of claim 6, wherein said composition is administered systemically.

12. The method of claim 6, wherein said composition is administered orally.
